(19) United States
(12) Patent Application Publication
(10) Pub. No.: US 2010/0267127 A1
(43) Pub. Date: Oct. 21, 2010

(54) POLYMERASE CHAIN REACTION APPARATUS

(75) Inventors: Kwang Hyo CHUNG, Daejeon (KR); Yo Han CHOI, Daejeon (KR); JuHyun JEON, Daejeon (KR); Moon Youn JUNG, Daejeon (KR)

Correspondence Address: AMPACC Law Group, 3500 188th Street S.W., Suite 103, Lynnwood, WA, 98037, US

(73) Assignee: Electronics and Telecommunications Research Institute, Daejeon (KR)

(21) Appl. No.: 12/484,797

(22) Filed: Jun. 15, 2009

(30) Foreign Application Priority Data
Apr. 16, 2009 (KR) 10-2009-0032982

(51) Int. Cl. C12M 1/02

(52) U.S. Cl. 435/305.2; 435/289.1

(57) ABSTRACT

Provided is a polymerase chain reaction (PCR) apparatus. A PCR is performed using the module assembly-type PCR apparatus. The module assembly-type PCR apparatus includes a first module, a second module, and a third module. A sample is provided to the first module. The second module provides different temperature ranges to the first module to generate thermal convection. The third module controls an operation of the second module. The first module is separably coupled to the second module. The second module is electrically separably coupled to the third module.

### CROSS-REFERENCE TO RELATED APPLICATIONS

This U.S. non-provisional patent application claims priority under 35 U.S.C. §119 of Korean Patent Application No. 10-2009-032982, filed on Apr. 16, 2009, the entire contents of which are hereby incorporated by reference.

### BACKGROUND

The present invention disclosed herein relates to a polymerase chain reaction apparatus, and more particularly, to a polymerase chain reaction apparatus using natural convection.

Generally, in biochips, biosensors, and chemical sensors that are used for biochemical analysis, a sample temperature is changed to a certain temperature to cause the sample to a predetermined reaction or increase reaction efficiency. Thus, to change the sample temperature to a suitable temperature, various heating methods are being proposed. A DNA amplification method using a polymerase chain reaction (hereinafter, referred to as a “PCR”) is a typical example of a biochemical reaction method in which a predetermined reaction is performed through a certain temperature change. The PCR is a type of the DNA amplification method in which a certain temperature cycling is performed on a sample prepared by mixing a DNA template, a primer, and an enzyme to increase the number of a target DNA through a catalytic chain reaction.

The temperature cycling is to change a temperature to two or three different degrees. As previously well-known, the PCR includes three processes. That is, the PCR includes a denaturation or degeneration process for separating a DNA double helix, an annealing or coupling process for controlling the DNA template to allow the primer to find a complementary pair, and an extension or polymerization process for growing the DNA. The temperature cycling is to sequentially change the sample temperature to temperatures different from each other.

The heating methods for changing the PCR temperature may be classified into two categories. The first method is to change a temperature of a sample by externally applying a temperature change to the sample. The second method is to change a temperature of a sample by moving the sample to an environment maintained at predetermined temperatures different from each other. The first method does not require an additional fluid control operation because the sample is not moved. The second method can rapidly change the temperature of the sample because the external temperature can be constantly maintained. However, in the first method, a lot of time is taken to change the temperature when a thermal capacity of the external environment is large. In addition, a control operation for the temperature change is additionally required. Thus, the control operation becomes complicated. Also, the second method requires a fluid control operation for moving the sample.

### SUMMARY

The present invention provides a polymerase chain reaction (PCR) apparatus in which a control operation and a fluid control operation for changing a temperature can be omitted by utilizing natural convection.

The present invention also provides a PCR apparatus in which modules can be easily replaced and a PCR condition change in each of the modules can be free because the PCR apparatus has a mutually separable module assembly structure.

The present invention also provides a PCR apparatus that can be miniaturized and portable because the PCR apparatus utilizes natural convection without requiring an external flow control.

Embodiments of the present invention provide polymerase chain reaction (PCR) apparatuses including: a second module separably coupled to a first module to which a sample is provided, the second module providing different temperature ranges to the first module to generate thermal convection; and a third module coupled to the second module, the third module controlling an operation of the second module.

In some embodiments, the first module may include a loop channel providing a loop type flow path of the sample that flows by thermal convection.

In other embodiments, the second module may include a plurality of heating parts that provides the different temperature ranges to the loop channel.

In still other embodiments, the plurality of heating parts may be disposed along the loop channel to provide heat having the different temperature ranges to portions of the loop channel.

In even other embodiments, the first module may be coupled to the second module in a state where the first module is inclined at a certain angle with respect to a gravitational direction.

In yet other embodiments, the third module may control whether heat having the different temperature range is provided.

In further embodiments, the second module may be electrically separably coupled to the third module.

In other embodiments of the present invention, PCR apparatuses include a chip including a loop channel that provides a loop type flow path of a sample; a unit module to which the chip is separably coupled, the unit module including a plurality of heating parts disposed along the loop channel to provide heat having different temperature ranges to the loop channel, thereby causing a loop-type flow of the sample along the loop channel by natural convection generated by the provided heat; and a mother module electrically connected to the unit module, the mother module controlling heating temperatures of the plurality of heating parts.

In some embodiments, the chip may include: a first plate including the loop channel; and a second plate coupled to the first plate to cover the loop channel, the second plate including a sample injection hole and a sample discharge hole that are connected to the loop channel.

In other embodiments, the plurality of heating parts may include: a first heating part heated to a temperature range required for a denaturation process of a PCR; a second heating part heated to a temperature range required for an annealing process of the PCR; and a third heating part heated to a temperature range required for a extension process of the PCR.

In still other embodiments, at least one of the first to third heating parts may include a metal heating plate in which a heater is disposed between stacked metal plates.

In even other embodiments, the metal plates may further include contact parts contacting with the loop channel, respectively, and the contact parts may be spaced by a space adapted to insert the chip therein.

In yet other embodiments, the at least one of the first to third heating parts may further include a temperature sensor that measures a temperature of the metal heating plate.

In further embodiments, the temperature sensor may be disposed in one of the contact parts.

In still further embodiments, the metal plates may further include insertion portions in which the heater is inserted, respectively.

In even further embodiments, the unit module may include: a housing in which the plurality of heating parts is built, the housing including a first connector to which the mother module is electrically connected and an insertion hole in which the chip is inserted; and a cover including an elastic plate covering the housing and sealing the loop channel.

In yet further embodiments, the housing may further include a partition therein, wherein the plurality of heating parts is disposed spaced apart from each other on a surface of the partition facing the cover, and a temperature measurement board measuring temperatures of the plurality of heating parts is disposed on an opposite surface of the partition.

In yet further embodiments, the plurality of heating parts may be spaced apart from the surface of the partition.

In yet further embodiments, the mother module may include: a second connector electrically connected to the first connector; and a temperature control board controlling heating temperatures of the plurality of heating parts.

In yet further embodiments, the mother module may be connected to at least two or more unit modules, wherein a PCR may be performed in each of the at least two unit modules.

In yet further embodiments, the mother module may be connected to at least two unit modules, wherein a PCR may be performed in one of the at least two unit modules as a first condition, and the PCR may be performed in the other unit module as a second condition equal to or different from the first condition.

In other embodiments of the present invention, method for performing a PCR use a PCR apparatuses including: a second module separably coupled to a first module to which a sample is provided, the second module providing different temperature ranges to the first module to generate thermal convection; and a third module coupled to the second module, the third module controlling an operation of the second module. The methods for performing the PCR include: coupling the first module to which the sample is provided to the second module; controlling the second module using the third module to provide heat having the different temperature ranges to the first module; and changing a temperature of the sample by the thermal convection due to the provided heat.

In some embodiments, at least two second modules may be connected to the third module to independently perform the PCR in each of the at least two second modules.

### BRIEF DESCRIPTION OF THE FIGURES

The accompanying figures are included to provide a further understanding of the present invention, and are incorporated in and constitute a part of this specification. The drawings illustrate exemplary embodiments of the present invention and, together with the description, serve to explain principles of the present invention. In the figures:

FIG. 1 is a perspective view of a polymerase chain reaction (PCR) apparatus according to an embodiment of the present invention;

FIG. 2 is an enlarged exploded perspective view illustrating a polymer chip of a PCR apparatus according to an embodiment of the present invention;

FIG. 3 is a perspective view illustrating a unit module of a PCR apparatus according to an embodiment of the present invention;

FIG. 4 is an exploded perspective view illustrating a coupling state of a first heating part and a chip of a PCR apparatus according to an embodiment of the present invention;

FIG. 5 is a cross-sectional view illustrating a coupling state of a first heating part and a chip of a PCR apparatus according to an embodiment of the present invention;

FIG. 6 is a cross-sectional view of a unit module in which a chip is inserted in a PCR apparatus according to an embodiment of the present invention;

FIG. 7 is a front view illustrating a coupling state of a chip and heating parts in a PCR apparatus according to an embodiment of the present invention;

FIGS. 8 and 9 are cross-sectional views illustrating examples of a PCR cycling in a polymerase chain reaction apparatus according to an embodiment of the present invention; and

FIG. 10 is a perspective view of a PCR apparatus according to another embodiment of the present invention.

### DETAILED DESCRIPTION OF EMBODIMENTS

Hereinafter, a polymerase chain reaction apparatus according to the present invention will be described in detail with reference to the accompanying drawings.

Advantages of the present invention in comparison with the related art will be clarified through the Detailed Description of Preferred Embodiments and the Claims with reference to the accompanying drawings. In particular, the present invention is well pointed out and clearly claimed in the Claims. The present invention, however, may be best appreciated by referring to the following Detailed Description of Preferred Embodiments with reference to the accompanying drawings. In the drawings, like reference numerals refer to like elements throughout.

### An Embodiment

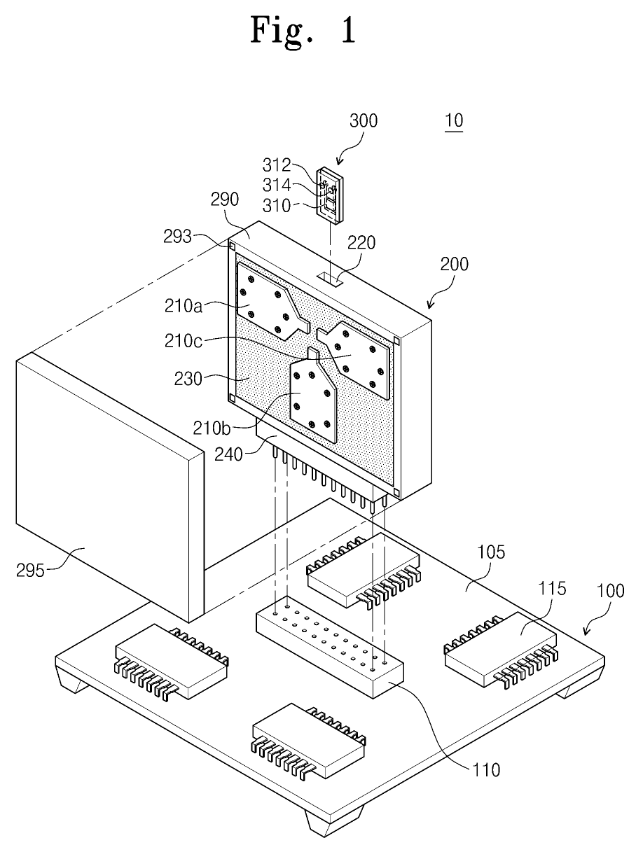

FIG. 1 is a perspective view of a polymerase chain reaction apparatus according to an embodiment of the present invention.

Referring to FIG. 1, a polymerase chain reaction apparatus 10 according to an embodiment of the present invention is an apparatus that can perform a polymerase chain reaction (hereinafter, referred to as a “PCR”) using a convection phenomenon. According to this embodiment, the PCR apparatus 10 may have a module assembly-type structure. For example, the PCR apparatus 10 may include a first module 300, a second module 200, and a third module 100. A sample may be injected and discharged through the first module 300. The second module 200 may provide heat having temperatures different from each other to the sample injected into the first module 300. The third module 100 may be electrically connected to the second module 200 to control the PCR. The sample may include a liquid sample. The first, second, and third modules 300, 200, and 100 may independently operate from each other. Thus, in case where one of the first to third modules 300, 200, and 100 causes a malfunction or is replaced, it may be easily replaced, separated, and coupled. The first module 300 may have a chip structure including a channel through which the sample is supplied. In this application, the first module 300 will be referred to as a chip or a polymer chip. The PCR using natural convection is substantially performed in the second module 200. The second module 200 will be referred to as a unit module. The third module 100 serves as a mother board. In this application, the third module 100 will be referred to as a mother module. In this application, the convection or natural convection denotes thermal convection that is naturally generated in a gravitational field by a fluid density difference due to a temperature difference. The thermal convection is distinguished from forced convection that is generated by forcedly moving a fluid using a pump or a propeller.

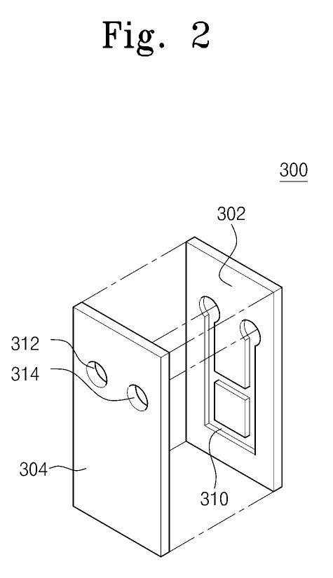

FIG. 2 is an enlarged exploded perspective view illustrating a polymer chip of a PCR apparatus according to an embodiment of the present invention.

Referring FIGS. 1 and 2, a first plate 302 and a second plate 304 may be coupled to form the chip 300. A loop channel 310 in which a loop-type flow of the sample is realizable by the convection may be disposed in the first plate 302. A sample injection hole 312 through which the sample is injected and a sample discharge hole 314 through which the sample is discharged may be defined in the second plate 304. The first plate 302 and the second plate 304 may be coupled to cover the loop channel 310 by the second plate 304. The first plate 302 and the second plate 304 may be coupled using an adhesive or a screw. The sample may be provided into the loop channel 310 by an injector or a capillary phenomenon. Although the loop channel 310 has a substantially square shape in FIG. 2, the present invention is not limited thereto. For example, the loop channel 310 may have a polygonal, circular, or oval shape. The first plate 302 and the second plate 304 may be formed of a certain material in which a thermal conductivity is relatively low and a thermal interference effect is minimized, i.e., may be formed of a polymer material such as polymethyl-methacrylate (PMMA) or poly carbonate (PC). Alternatively, the first plate 302 and the second plate 304 may be formed of silicon. The chip 300 formed of a polymer will be described in this embodiment. The term “chip 300” will be used together with a term “polymer chip” in this application. The polymer chip 300 may be disposable or reusable. The loop channel 310 may be surface-treated to prevent components such as biomolecules within the liquid sample from being absorbed to the loop channel 310. For example, the loop channel 310 may be exposed to plasma so that a surface of the loop channel 310 has a hydrophobic or hydrophilic property.

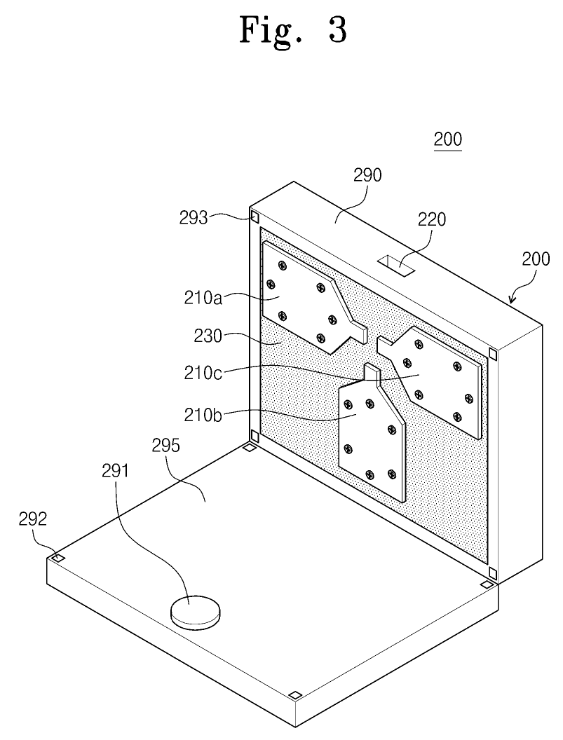

FIG. 3 is a perspective view illustrating a unit module of a PCR apparatus according to an embodiment of the present invention.

Referring to FIGS. 1 and 3, the unit module 200 may realize the loop-type flow of the sample by the convection phenomenon. For example, the unit module 200 may include a plurality of heating parts 210a, 210b, and 210c and a housing 290. The plurality of heating parts 210a through 210c may be heated at temperatures different from each other. The housing 290 includes a built-in board 230 (hereinafter, referred to as a “temperature measurement board”) that can measure the temperatures of the plurality of heating parts 210a, 210b, and 210c. The housing 290 may have a closed or opened structure. According to this embodiment, the housing 290 may have a substantially hexahedral or polyhedral shape having an opened surface. In case where the housing 290 has the opened structure, the unit module 200 may further include a cover 295 that can cover the housing 290. The housing 290 may include an insertion hole 220 having a slot shape and a connector 240. The polymer chip 300 is inserted into the insertion hole 220. The connector 240 electrically connects the unit module 200 to the mother module 100. According to this embodiment, the insertion hole 220 may be defined in a top surface of the housing 290, and the connector 240 may be disposed on a bottom surface of the housing 290.

The first to third heating parts 210a, 210b, and 210c may be heated to temperature ranges required for three PCR processes. For example, the first heating part 210a, the second heating part 210b, and the third heating part 210c may be heated to the temperature ranges required for a denaturation process, an extension process, and an annealing process, respectively. As another example, the first heating part 210a, the second heating part 210b, and the third heating part 210c may be heated to the temperature ranges required for the annealing process, the extension process, and the denaturation process, respectively. As further another example, the first heating part 210a, the second heating part 210b, and the third heating part 210c may be heated to the temperature ranges required for the annealing process, the denaturation process, and the extension process, respectively. As yet another example, the first heating part 210a, the second heating part 210b, and the third heating part 210c may be heated to the temperature ranges required for the extension process, the denaturation process, and the annealing process, respectively. The temperature for the denaturation process may range from about 90° C. to about 97° C. The temperature for the annealing process may range from about 50° C. to about 65° C. The temperature for the extension process may range from about 68° C. to about 74° C.

The first to third heating parts 210a, 210b, and 210c may be disposed in a loop shape to sequentially perform the denaturation, annealing, and extension processes on the sample. According to this embodiment, the first and third heating parts 210a and 210c may be disposed along a horizontal line, and the second heating part 210b may be disposed along a vertical line. The second heating part 210b may be disposed at about 90° with respect to the first and third heating part 210a and 210c. For example, the first heating part 210a may be disposed on an upper left portion of the board 230, the second heating part 210b may be disposed on a lower central portion of the board 230, and the third heating part 210c may be disposed on an upper right portion. Thus, the sample supplied into the loop channel 310 may be sequentially set to the temperature ranges different from each other to realize the PCR cycling. The PCR cycling may be realized by the natural convection due to the temperature difference transferred from the first to third heating parts 210a, 210b, and 210c without applying an external force.

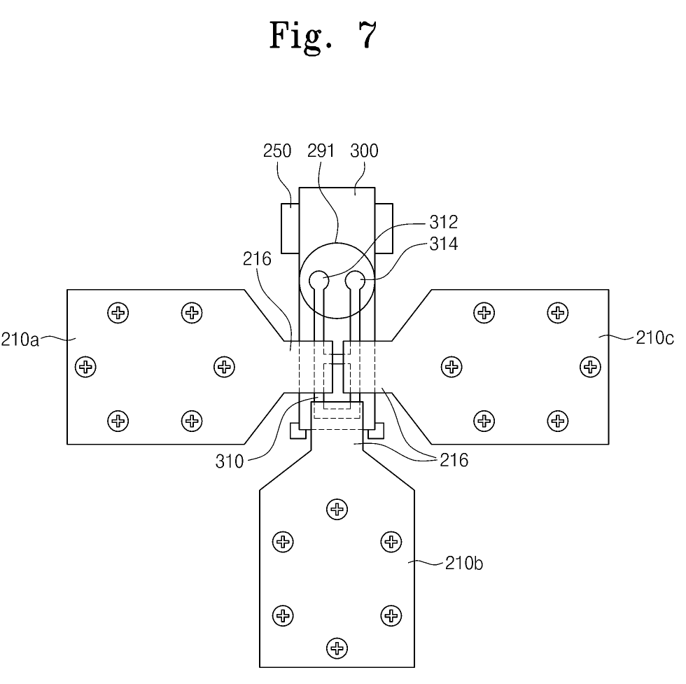

The cover 295 may be hinge-coupled to the housing 290. An elastic plate 291 may be disposed inside the cover 295 facing the housing 290. The elastic plate 291 may be disposed at a position covering the sample injection hole 312 and the sample discharge hole 314 when the cover 295 is closed. As described below with reference to FIG. 7, when the cover 295 is closed to cover the housing 290, the elastic plate 291 may be elastically deformed to cover the sample injection hole 312 and the sample discharge hole 314 of the polymer chip 300, thereby sealing the loop channel 310. As described above, the PCR process may include the denaturation process performed under a high temperature condition over about 90° C. As a result, the liquid sample circulating in the loop channel 310 under an atmospheric pressure may be boiled to generate bubbles. The bubbles may interrupt the PCR. However, since the loop channel 310 is sealed by the elastic plate 291, a pressure within the loop channel 310 may increase to increase a boiling point even if an internal environment of the loop channel 310 is in a high temperature state. Thus, although the internal temperature of the loop channel 310 is over about 90° C., the bubbles may not be generated. Magnets 292 may be disposed in inner edges of the cover 295, and magnets 293 may be disposed in inner edges of the housing 290 corresponding to the positions of the magnets 292. The magnets 292 and the magnets 293 that correspond to each other may have opposite polarities from each other to provide an attractive force therebetween. When the cover 295 is closed, the cover may be strongly coupled to the housing 290 due to the attractive force between the magnets 292 and the magnets 293. Alternatively, the cover 295 may be screw-coupled to the housing 290.

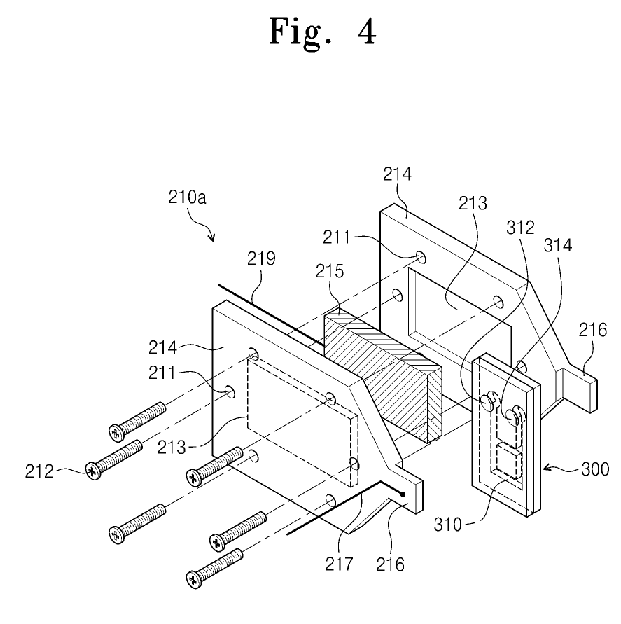

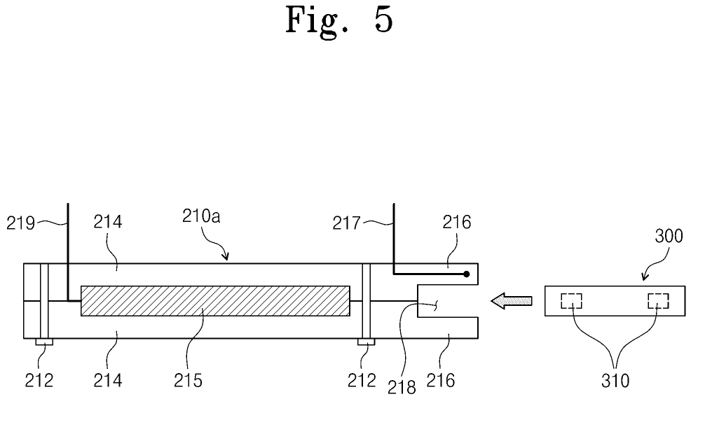

FIG. 4 is an exploded perspective view illustrating a coupling state of a first heating part and a chip of a PCR apparatus according to an embodiment of the present invention, and FIG. 5 is a cross-sectional view illustrating a coupling state of a first heating part and a chip of a PCR apparatus according to an embodiment of the present invention. The following descriptions with respect to the first heating part 210a may also be applicable to the second and third heating parts 210b and 210c.

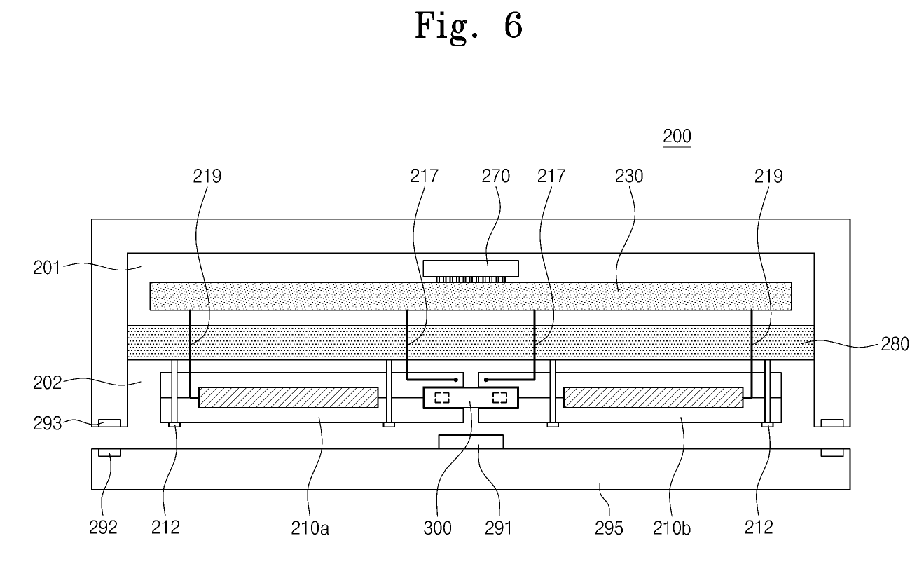

Referring to FIGS. 4 and 5, the first heating part 210a may include a metal heating plate. For example, the first heating part 210a may have a stacked structure in which a heater 215 is disposed between two heating plates 214. The heating plates 214 may be formed of a material having a high thermal conductivity, e.g., a metal such as gold, silver, platinum, copper, or an alloy thereof. Thus, the heating plates 214 may provide a uniform temperature and faster thermal conduction. The heating plates 214 may be screw-coupled to each other. For example, screw holes 211 may be defined in the heating plates 214, and screws 212 may be inserted into the screw holes 211 to couple the heating plates 214 to each other. Each of the screws 212 may have a length corresponding to a depth of the heating plates 214. As another example, as described below with reference to FIG. 6, each of the screws 212 may have a length longer than the depth of the heating plates 214. Thus, the heating plates 214 may be coupled to each other, as well as the first heating part 210a may be fixed to the housing 290. The heater 215 may have one end connected to a heat wire 219 through which a current is applied to the heater 215. The heater 215 may include one of a film heater, a ceramic heater, and a rod heater.

Insertion portions 213 in which the heater 215 is inserted may be provided in inner surfaces of the heating plates 214. Each of the insertion portions 213 may have a thickness and width approximately corresponding to those of each of the heating plates 214 to efficiently transfer heat from heater 215 toward the heating plates 214. In addition, thermal paste or thermal grease may be additionally filled into spaces between the heater 215 and the heating plates 214 to increase the thermal conduction. The first heating part 210a may provide a space 218 in which the polymer chip 300 is inserted between the heating plates 214. For example, contact parts 216 may be provided at one ends of the heating plates 214, and the polymer chip 300 may be inserted into the space 218 between the contact parts 216. Thus, the polymer chip 300 may receive heat from both contact parts 216. The contact parts 216 may be spaced at least by a thickness of the polymer chip 300. Accordingly, the polymer chip 300 may be in contact with the contact parts 216 without a gap to efficiently transfer heat from the contact parts 216 toward the polymer chip 300. Also, to increase the thermal conduction between the contact parts 216 and the polymer chip 300, the thermal paste or the thermal grease may be coated on inner surfaces of the contact parts 216 contacting with the polymer chip 300. The polymer chip 300 may have a tapered shape which is tapered in an insertion direction (in an arrow direction of FIG. 5) to easily insert the polymer chip 300 into the space 218 between the contact parts 216. Also, each of the contact parts 216 may have a shape corresponding to the tapered shape of the polymer chip 300 to minimize gaps between the contact parts 216 and the polymer chip 300, thereby increasing the thermal conduction.

The first heating part 210a may include a temperature sensor 217 that detects a temperature thereof. For example, the temperature sensor 217 may be disposed at a position most adjacent to the polymer chip 300, e.g., at any one contact part 216 to relatively accurately measure the temperature transferred into the polymer chip 300. For example, a portion of any one contact part 216 may be punched or hollow out a groove to install the temperature sensor 217 inside the contact part 216. A portion of any one heating plate 214 may be punched or hollow out a groove to extend a heater wire 219 to the outside of the first heating part 210a.

FIG. 6 is a cross-sectional view of a unit module in which a chip is inserted in a PCR apparatus according to an embodiment of the present invention.

Referring to FIGS. 1 and 6, a partition 280 for separating a space within the housing 290 into at least two regions 201 and 202 may be disposed in the housing 290. A temperature measurement board 230 may be disposed in one region 201 of the two regions 201 and 202. The first to third heating parts 201a, 201b, and 201c may be disposed in the other region 202. The first to third heating parts 201a, 201b, and 201c may be fixed to one surface of the partition 280 facing the cover 295 using the screws 212. The temperature measurement board 230 may be fixed to the other surface of the partition 280 using a fixing unit such as the screw or the adhesive. The temperature measurement board 230 is spatially spaced apart from the first to third heating parts 210a, 210b, and 210c, but may be electrically connected to the first to third heating parts 210a, 210b, and 210c. For example, the heater wires 219 and the temperature sensors 217 of the first to third heating parts 210a, 210b, and 210c may be connected to the temperature measurement board 230. Thus, heating temperatures of the first to third heating parts 210a, 210b, and 210c may be controlled by the temperature measurement board 230. Each of the temperature sensors 217 may have a thermocouple structure using an electromotive force due to a temperature difference between a reference temperature and a measured temperature. In this case, the reference temperature may be influenced by an ambient temperature. Thus, the reference temperature may be corrected according to the ambient temperature to relatively accurately indicate the measured temperature, i.e., the temperatures of the first to third heating parts 210a, 210b, and 210c. Accordingly, a semiconductor chip 270 that measures the reference temperature may be further disposed on the temperature measurement board 230.

The first to third heating parts 210a, 210b, and 210c may be spaced from each other and fixed to the partition 280 to maintain constant temperatures or temperature ranges different from each other and prevent thermal interference therebetween. In addition, the first to third heating parts 210a, 210b, and 210c may be spaced from the partition 280 to minimize the thermal conduction from the first to third heating parts 210a, 210b, and 210c toward the partition 280. Since the thermal conduction from the first to third heating parts 210a, 210b, and 210c toward the partition 280 occurs by the screws 212, each of the screws 212 may be formed of a polymer having a relatively good thermal insulation property. Also, since heat generated in the first to third heating parts 210a, 210b, and 210c is transferred to the temperature measurement board 230 to cause the malfunction of the temperature measurement board 230, the partition 280 may be formed of a polymer having a relatively low thermal conductivity.

The density of the flow of the natural convection may be proportional to the strength of the gravitational field. Thus, when the polymer chip 300 is inclined at a certain angle with respect to a gravitational field direction, a flow velocity of the natural convection may be slow to increase a flow time of the PCR. For example, In case where the polymer chip 300 is perpendicular to the gravitational field direction, i.e., 90° C., the flow time of the PCR may become slower when compared to a case in which the polymer chip 300 is disposed on a straight line extending in the gravitational field direction, i.e., 0° C. In the unit module 200, the polymer chip 300 may be inclined at a predetermined angle with respect to the gravitational field direction to adjust the flow time of the PCR. The unit module 200 may optionally include an optical detection module or an electrical detection module for detecting the PCR process and/or the PCR results in real time.

FIG. 7 is a front view illustrating a coupling state of a chip and heating parts in a PCR apparatus according to an embodiment of the present invention.

Referring to FIGS. 1 and 7, a left portion of the loop channel 310 of the polymer chip 300 may be in contact with the contact part 216 of the first heating part 210a. A lower portion of the loop channel 310 may be in contact with the contact part 216 of the second heating part 210b. A right portion of the loop channel 310 may be in contact with the contact part 216 of the third heating part 210c. A slot-type guide 250 for guiding the insertion of the polymer chip 300 may be further provided in the insertion hole 220 to contact the contact parts 216 with the loop channel 210 as described above. The guide 250 may be provided in plurality at positions corresponding to lateral edges and lower end edges of the polymer chip 300 on an inner wall of the insertion hole 220. As described above with reference to FIG. 3, when the PCR cycling is performed, the elastic plate 291 may cover the sample injection hole 312 and the sample discharge hole 314 of the polymer chip 300 to seal the loop channel 310.

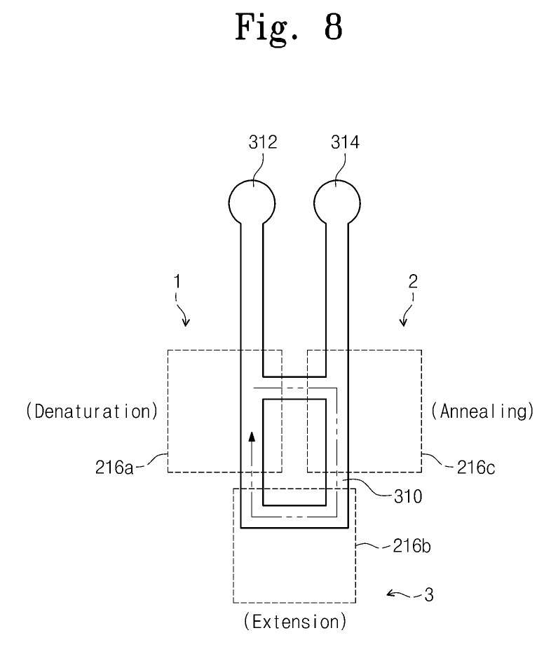

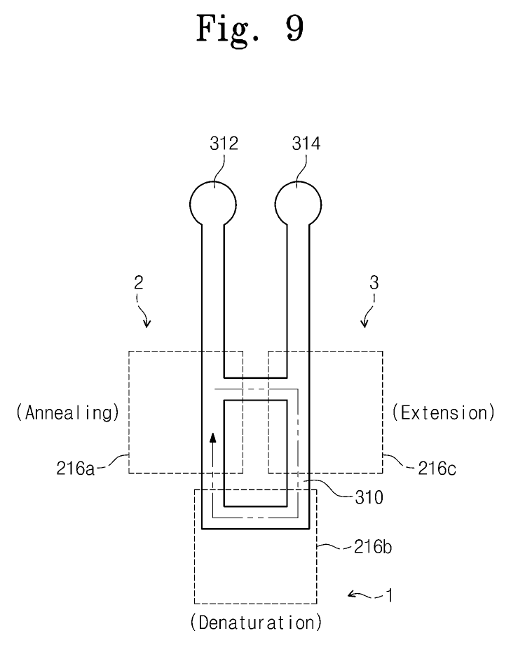

FIGS. 8 and 9 are cross-sectional views illustrating examples of a PCR cycling in a polymerase chain reaction apparatus according to an embodiment of the present invention. In the drawings, reference numerals 216a, 216b, and 216c denote the contact parts of the first to third heating parts 210a, 210b, and 210c, respectively. For convenience of distinction, the contact part of the first heating part 210a will be referred to as a first contact part 216a, the contact part of the second heating part 210b will be referred to as a second contact part 216b, and the contact part of the third heating part 210c will be referred to as a third contact part 216c.

Referring to FIG. 8, according to an exemplary embodiment of the present invention, a left portion of the loop channel 310 adjacent to the sample injection hole 312 may be in contact with the first contact part 216a. A lower portion of the loop channel 310 may be in contact with the second contact part 216b. A right portion of the loop channel 310 adjacent to the sample discharge hole 314 may be in contact with the third contact part 216c. The first contact part 216a and/or the third contact part 216c may have a size capable of being in contact with horizontal and vertical portions of the loop channel 310 or a size capable of being in contact with the vertical portion of the loop channel 310. Similarly, the second contact part 216b may have a size capable of being in contact with the horizontal and vertical portions of the loop channel 310 or a size capable of being in contact with the horizontal portion of the loop channel 310. For example, the first contact part 216a may be heated to a high temperature range (for example, about 90° C. to about 97° C.) required for the denaturation process. The second contact part 216b may be heated to a middle temperature range (about 68° C. to about 74° C.) required for the extension process. The third contact part 216c may be heated to a low temperature range (about 50° C. to about 65° C.) required for the annealing process. Thus, the loop channel 310 may be divided into a high temperature region 1 contacting with the first contact part 216a, a middle temperature region 3 contacting with the second contact part 216b, and a low temperature region 2 contacting with the third contact part 216c.

The liquid sample provided into the loop channel 210 that is divided into the temperature regions 1, 2 and 3 different from each other may flow in a clockwise direction by a buoyant force generated by a density difference due to thermal convection to perform the PCR cycling. For example, the denaturation process may be performed on the liquid sample provided into the loop channel 310 in the high temperature region 1 to separate double-stranded DNA into single-stranded DNA. The annealing process may be performed in the low temperature region 2 to couple single-stranded DNA to a primer having a base sequence complementary to the single stranded DNA. The extension process may be performed in the middle region 3 to growth DNA. This PCR cycling is repeated once or several times to amplify the DNA. The results of the PCR cycling may be detected using a fluorescent material in real time. As another example, the high temperature region 1 and the low temperature region 2 may be changed in their position with each other. Thus, the sample may flow in a counterclockwise direction to realize the PCR cycling.

Referring to FIG. 9, according to another exemplary embodiment of the present invention, the first contact part 216a may be heated to the low temperature range required for the annealing process. The second contact part 216b may be heated to the high temperature range required for the denaturation process. The third contact part 216c may be heated to the middle temperature range required for the extension process. Thus, a lower portion, a left portion, and a right portion of the loop channel 310 may be defined as a high temperature region 1, a low temperature region 2, and a middle temperature region 3, respectively. The flow velocity of the sample may gradually increase from the high temperature region 1 toward the low temperature region 2 to realize the PCR cycling in a clockwise direction. As another example, the low temperature region 2 and the middle temperature region 3 may be changed in their position with each other. Thus, the sample may flow in a counterclockwise direction to realize the PCR cycling.

Again referring to FIG. 1, the mother module 100 may include a board 105 (hereinafter, referred to as a “temperature control board”) for controlling the temperatures of the first to third heating parts 210a, 210b, and 210c and a connector 110 in which the connector 240 of the unit module 200 is inserted. The temperature control board 105 may include one or more semiconductor chip 115 for controlling an operation of the mother module 100.

### Another Embodiment

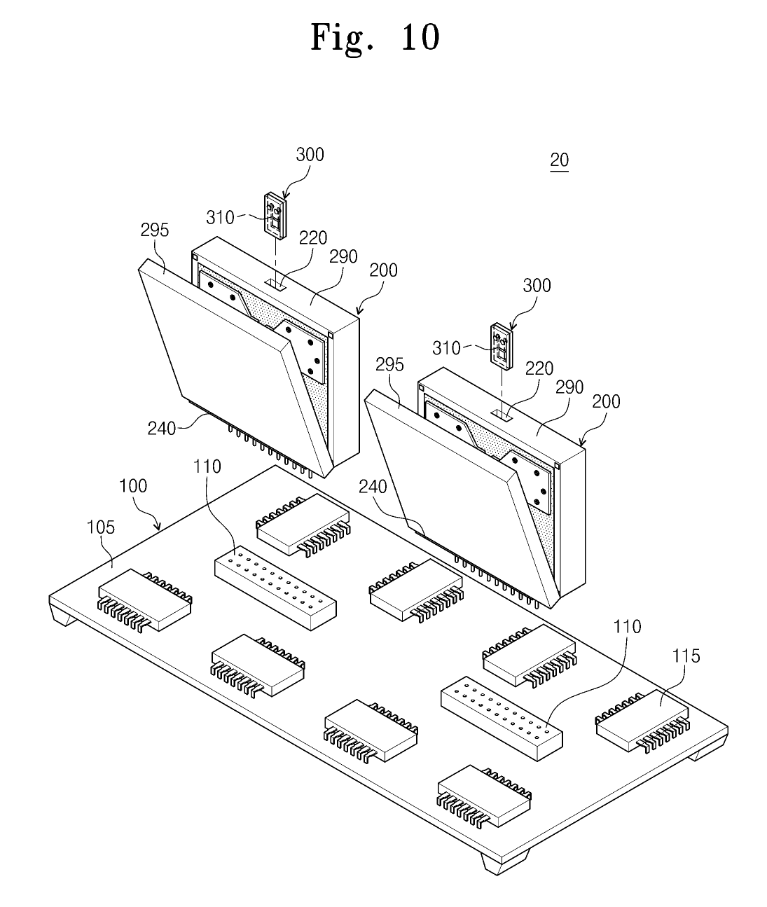

FIG. 10 is a perspective view of a PCR apparatus according to another embodiment of the present invention. Since this embodiment is similar to that described previously, only the differences from the previously described embodiment will be described and the other will be omitted.

Referring to FIG. 10, a PCR apparatus 20 according to this embodiment may perform a plurality of PCR cyclings at the same time. For example, the PCR apparatus 20 may include two unit modules 200 electrically connected to one mother module 100. A polymer chip 300 may be inserted into each of the two unit modules 200. According to this embodiment, PCR conditions may be freely changed in each of the unit modules 200. For example, a PCR may be performed in one of the two unit modules 200 as a first condition, and the PCR may be performed in the other unit module 200 as a second condition equal to or different from the first condition. The different condition may denote a condition that DNAs to be amplified are different from each other or a condition that temperature ranges of the PCR cyclings performed in one unit module 200 are different from those of the PCR cyclings performed in the other unit module 200.

According to the present invention, since the PCR apparatus using natural convection is modularized, a device for controlling the temperature change and fluid of the fluid is not required. Therefore, the PCR apparatus can be miniaturized, and the modules can be easily replaced. In addition, the PCR condition can be freely changed.

The above-disclosed subject matter is to be considered illustrative, and not restrictive, and the appended claims are intended to cover all such modifications, enhancements, and other embodiments, which fall within the true spirit and scope of the present invention. Thus, to the maximum extent allowed by law, the scope of the present invention is to be determined by the broadest permissible interpretation of the following claims and their equivalents, and shall not be restricted or limited by the foregoing detailed description.

What is claimed is:

1. A polymerase chain reaction (PCR) apparatus comprising:
a second module separably coupled to a first module to which a sample is provided, the second module providing different temperature ranges to the first module to generate thermal convection; and
a third module coupled to the second module, the third module controlling an operation of the second module.

2. The PCR apparatus of claim 1, wherein the first module comprises a loop channel providing a loop type flow path of the sample that flows by thermal convection.

3. The PCR apparatus of claim 2, wherein the second module comprises a plurality of heating parts that provides the different temperature ranges to the loop channel.

4. The PCR apparatus of claim 3, wherein the plurality of heating parts is disposed along the loop channel to provide heat having the different temperature ranges to portions of the loop channel.

5. The PCR apparatus of claim 1, wherein the first module is coupled to the second module in a state where the first module is inclined at a certain angle with respect to a gravitational direction.

6. The PCR apparatus of claim 1, wherein the third module controls whether heat having the different temperature range is provided.

7. The PCR apparatus of claim 1, wherein the second module is electrically separably coupled to the third module.

8. A polymerase chain reaction (PCR) apparatus comprising:
a chip comprising a loop channel that provides a loop type flow path of a sample;
a unit module to which the chip is separably coupled, the unit module comprising a plurality of heating parts disposed along the loop channel to provide heat having different temperature ranges to the loop channel, thereby causing a loop-type flow of the sample along the loop channel by natural convection generated by the provided heat; and
a mother module electrically connected to the unit module, the mother module controlling heating temperatures of the plurality of heating parts.

9. The PCR apparatus of claim 8, wherein the chip comprises:
a first plate comprising the loop channel; and
a second plate coupled to the first plate to cover the loop channel, the second plate comprising a sample injection hole and a sample discharge hole that are connected to the loop channel.

10. The PCR apparatus of claim 8, wherein the plurality of heating parts comprises:
a first heating part heated to a temperature range required for a denaturation process of a PCR;
a second heating part heated to a temperature range required for an annealing process of the PCR; and
a third heating part heated to a temperature range required for a extension process of the PCR.

11. The PCR apparatus of claim 10, wherein at least one of the first to third heating parts comprises a metal heating plate in which a heater is disposed between stacked metal plates.

12. The PCR apparatus of claim 11, wherein the metal plates further comprise contact parts contacting with the loop channel, respectively, and the contact parts is spaced by a space adapted to insert the chip therein.

13. The PCR apparatus of claim 12, wherein the at least one of the first to third heating parts further comprises a temperature sensor that measures a temperature of the metal heating plate.

14. The PCR apparatus of claim 13, wherein the temperature sensor is disposed in one of the contact parts.

15. The PCR apparatus of claim 11, wherein the metal plates further comprise insertion portions in which the heater is inserted, respectively.

16. The PCR apparatus of claim 8, wherein the unit module comprises:
a housing in which the plurality of heating parts is built, the housing comprising a first connector to which the mother module is electrically connected and an insertion hole in which the chip is inserted; and
a cover comprising an elastic plate covering the housing and sealing the loop channel.

17. The PCR apparatus of claim 16, wherein the housing further comprises a partition therein,
wherein the plurality of heating parts is disposed spaced apart from each other on a surface of the partition facing the cover, and a temperature measurement board measuring temperatures of the plurality of heating parts is disposed on an opposite surface of the partition.

18. The PCR apparatus of claim 17, wherein the plurality of heating parts is spaced apart from the surface of the partition.

19. The PCR apparatus of claim 16, wherein the mother module comprises:
a second connector electrically connected to the first connector; and
a temperature control board controlling heating temperatures of the plurality of heating parts.

20. The PCR apparatus of claim 8, wherein the mother module is connected to at least two unit modules,
wherein a PCR is performed in one of the at least two unit modules as a first condition, and the PCR is performed in the other of the at least two unit module as a second condition equal to or different from the first condition.
